(19) United States
(12) Patent Application Publication
(10) Pub. No.: US 2006/0142477 A1
(43) Pub. Date: Jun. 29, 2006

(54) Method for making sulfoalkylated cellulose polymer network

(76) Inventors: Wolfgang G. Glasser, Blacksburg, VA (US); Alena Michalek, Auburn, WA (US); S. Ananda Weerawarna, Seattle, WA (US); Carole W. Herriott, Bremerton, WA (US); Richard A. Jewell, Tacoma, WA (US)

Correspondence Address: WEYERHAEUSER COMPANY;INTELLECTUAL PROPERTY DEPT., CH 1J27, P.O. BOX 9777, FEDERAL WAY, WA, 98063, US

(21) Appl. No.: 11/027,087

(22) Filed: Dec. 29, 2004

(51) Int. Cl. A61K 47/48

(52) U.S. Cl. 525/54.1

(57) ABSTRACT

Method for making a sulfoalkylated cellulose polymer network having superabsorbent properties.

### FIELD OF THE INVENTION

The present invention relates to methods for making a sulfoalkylated cellulose polymer network.

### BACKGROUND OF THE INVENTION

Personal care absorbent products, such as infant diapers, adult incontinent pads, and feminine care products, typically contain an absorbent core that includes superabsorbent polymer particles distributed within a fibrous matrix. Superabsorbents are water-swellable, generally water-insoluble absorbent materials having a high absorbent capacity for body fluids. Superabsorbent polymers (SAPs) in common use are mostly derived from acrylic acid, which is itself derived from oil, a non-renewable raw material. Acrylic acid polymers and SAPs are generally recognized as not being biodegradable. Despite their wide use, some segments of the absorbent products market are concerned about the use of non-renewable oil derived materials and their non-biodegradable nature. Acrylic acid based polymers also comprise a meaningful portion of the cost structure of diapers and incontinent pads. Users of SAP are interested in lower cost SAPs. The high cost derives in part from the cost structure for the manufacture of acrylic acid which, in turn, depends upon the fluctuating price of oil. Also, when diapers are discarded after use they normally contain considerably less than their maximum or theoretical content of body fluids. In other words, in terms of their fluid holding capacity, they are “over-designed”. This “over-design” constitutes an inefficiency in the use of SAP. The inefficiency results in part from the fact that SAPs are designed to have high gel strength (as demonstrated by high absorbency under load or AUL). The high gel strength (upon swelling) of currently used SAP particles helps them to retain a lot of void space between particles, which is helpful for rapid fluid uptake. However, this high “void volume” simultaneously results in there being a lot of interstitial (between particle) liquid in the product in the saturated state. When there is a lot of interstitial liquid the “rewet” value or “wet feeling” of an absorbent product is compromised.

In personal care absorbent products, U.S. southern pine fluff pulp is commonly used in conjunction with the SAP. This fluff is recognized worldwide as the preferred fiber for absorbent products. The preference is based on the fluff pulp's advantageous high fiber length (about 2.8 mm) and its relative ease of processing from a wetlaid pulp sheet to an airlaid web. Fluff pulp is also made from renewable and biodegradable cellulose pulp fibers. Compared to SAP, these fibers are inexpensive on a per mass basis, but tend to be more expensive on a per unit of liquid held basis. These fluff pulp fibers mostly absorb within the interstices between fibers. For this reason, a fibrous matrix readily releases acquired liquid on application of pressure. The tendency to release acquired liquid can result in significant skin wetness during use of an absorbent product that includes a core formed exclusively from cellulosic fibers. Such products also tend to leak acquired liquid because liquid is not effectively retained in such a fibrous absorbent core.

A need therefore exists for a superabsorbent material that is made from a biodegradable renewable resource like cellulose and that is inexpensive. In this way, the superabsorbent material can be used in absorbent product designs that are efficient such that they can be used closer to their theoretical capacity without feeling wet to the wearer. The present invention seeks to fulfill this need and provides further related advantages.

### SUMMARY OF THE INVENTION

The invention provides a method for making a sulfoalkyl cellulose polymer network having superabsorbent properties. In one embodiment, the method comprises reacting a sulfoalkyl cellulose and a synthetic water-soluble polymer with a crosslinking agent. The crosslinking agent reacts with at least one of the sulfoalkyl cellulose or synthetic water-soluble polymer. In another embodiment, the method comprises combining a sulfoalkyl cellulose, a synthetic water-soluble polymer, and a crosslinking agent in an aqueous solution to provide a polymer mixture; precipitating the polymer mixture by addition of a water-miscible solvent to provide a precipitated mixture; collecting the precipitated mixture; and crosslinking the precipitated mixture to provide the composition.

### BRIEF DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a cross sectional view of an absorbent construct incorporating sulfoalkylated cellulose of the invention and having an acquisition layer;.

FIG. 2 is a cross sectional view of an absorbent construct incorporating sulfoalkylated cellulose of the invention and having acquisition and distribution layers;

FIGS. 3A-C are cross sectional views of absorbent articles incorporating a composite including sulfoalkylated cellulose of the invention and the absorbent constructs illustrated in FIGS. 1 and 2, respectively; and

FIG. 4 is a schematic illustration of a device for measuring Absorbency Under Load (AUL) values.

### DETAILED DESCRIPTION OF THE PREFERRED EMBODIMENT

In one aspect, the invention provides a polymer network having superabsorbent properties. The network includes two types of polymers. The first polymer is a sulfoalkyl cellulose. The second polymer is a synthetic water-soluble polymer.

The polymer network (also referred to herein as “the composition” or “the superabsorbent composition”) is obtainable by reacting a sulfoalkyl cellulose and a synthetic water-soluble polymer with a crosslinking agent. In one embodiment, the sulfoalkyl cellulose and synthetic water-soluble polymer are reacted with two crosslinking agents. The crosslinking agent(s) reacts with at least one of the sulfoalkyl cellulose or synthetic water-soluble polymer to provide the network. In one embodiment, the polymer network is obtained by treating a sulfoalkyl cellulose and a synthetic water-soluble polymer with a crosslinking agent to provide a reaction mixture, and crosslinking the reaction mixture to provide the composition. In this embodiment, crosslinking the reaction mixture refers to crosslinking the sulfoalkyl cellulose, crosslinking the synthetic water-soluble polymer, and/or crosslinking the sulfoalkyl cellulose to the synthetic water-soluble polymer to provide the network.

In the network, the ratio of sulfoalkyl cellulose to synthetic water-soluble polymer is from about 50:50 to about 95:5 weight/weight. In one embodiment, the synthetic water-soluble polymer is present in the network in about 10 percent by weight based on the weight of the sulfoalkyl cellulose.

In certain embodiments, the polymer network of the invention includes two or more types of polymers.

In one embodiment, the sulfoalkyl cellulose is a water-soluble sulfoalkyl cellulose. In one embodiment, the sulfoalkyl cellulose is a sulfoethyl cellulose (also known as a cellulose ethyl sulfonate) made by cellulose sulfoalkylation with vinyl sulfonate or chloroethyl sulfonate, or their sulfonic acid derivatives. In one embodiment, the sulfoalkyl cellulose is a sulfo-2-hydroxypropyl cellulose (also known as a cellulose 2-hydroxypropyl sulfonate) made by cellulose sulfoalkylation with 3-chloro-2-hydroxypropyl sulfonate or chloroethyl sulfonate, or their sulfonic acid derivatives. Mixtures of sulfoalkyl celluloses can be used.

As used herein, the term “sulfoalkyl cellulose” or “sulfoalkylated cellulose” are used interchangeably and refer to cellulose that has been modified by alkylation with a sulfoalkylating agent to provide cellulose having pendant alkyl sulfonate groups. The sulfoalkyl cellulose is a cellulose ether in which cellulose hydroxy groups are etherified (i.e., alkylated) with alkyl sulfonate groups. The alkyl sulfonate groups are covalently coupled to cellulose through ether groups. As used herein, the term “sulfonate” refers to sulfonic acid and sulfonic acid salts, for example, sodium and potassium salts.

Sulfoalkyl cellulose can be obtained by alkylation (i.e., etherification) of cellulose (e.g., alkali cellulose) with suitable sulfoalkylating agents. Suitable sulfoalkylating agents include haloalkyl sulfonates and vinyl sulfonates (and their metal salts, e.g., sodium and potassium). Suitable haloalkyl sulfonates include chloroethyl sulfonate (CES), bromoethyl sulfonate (BES), and 3-chloro-2-hydroxypropyl sulfonate (CHPS). Chloroethyl sulfonate is commercially available from a variety of sources or can be prepared by the reaction of vinyl chloride and sodium bisulfite in alcohol solvent. 3-Chloro-2-hydroxypropyl sulfonate is also commercially available from a variety of sources or by reaction of epichlorohydrin with sodium bisulfite. Vinyl sulfonate (sodium form) is commercially available from a variety of sources.

Cellulosic fibers suitable for use in making the sulfoalkyl cellulose useful in making the polymer networks of the invention are substantially water insoluble and not highly water swellable. After sulfoalkylation, the resulting sulfoalkyl cellulose is water soluble.

As used herein, a material will be considered to be water soluble when it substantially dissolves in excess water to form a solution, losing its form and becoming essentially evenly dispersed throughout a water solution.

The sulfoalkyl cellulose polymer networks of the invention are water swellable and water insoluble. As used herein, the terms “water swellable” and “water insoluble” refer to a cellulose that, when exposed to an excess of an aqueous medium (e.g., bodily fluids such as urine or blood, water, synthetic urine, or 1 weight percent solution of sodium chloride in water), swells to an equilibrium volume, but does not dissolve into solution.

Suitable sulfoalkyl celluloses useful in making the polymer networks have an average degree of sulfonate group substitution of from about 0.05 to about 2.0. In one embodiment, the cellulose has an average degree of substitution of from about 0.1 to about 1.0. In another embodiment, the cellulose has an average degree of substitution of from about 0.3 to about 0.5. As used herein, the “average degree of sulfonate group substitution” refers to the average number of moles of sulfonate groups per mole of glucose unit in the polymer.

Suitable sulfoalkyl celluloses useful in making the polymer networks have viscosity (1 percent aqueous solution) of from about 10 to about 250 cP.

The sulfoalkyl celluloses useful in making the polymer networks can also be carboxyalkylated celluloses. Thus, in certain embodiments, the sulfoalkyl cellulose also includes carboxyalkyl groups. In certain embodiments, the sulfoalkyl cellulose includes carboxymethyl groups, carboxyethyl groups, or mixtures of carboxymethyl and carboxyethyl groups. As used herein, the term “sulfoalkyl cellulose” also refers to celluloses having both sulfoalkyl groups and carboxyalkyl groups. In certain embodiments, suitable sulfoalkyl celluloses useful in making the polymer networks have an average degree of carboxy group substitution of from about 0.01 to about 2.0. In one embodiment, the cellulose has an average degree of substitution of from about 0.1 to about 1.0. As used herein, the “average degree of carboxy group substitution” refers to the average number of moles of carboxy groups per mole of glucose unit in the polymer.

Sulfoalkyl cellulose can be made by treating alkalized cellulose with one or more sulfoalkylating agents. As mentioned above, the sulfoalkyl cellulose can be a carboxyalkyl cellulose, which can be made by treating alkalized cellulose with one or more sulfoalkylating agents and one or more carboxyalkylating agents.

In one embodiment, the method includes the following steps:

(a) treating cellulose with alkali to provide alkali cellulose;

(b) treating the alkali cellulose with a sulfoalkylating agent (or sulfoalkylating agent and carboxyalkylating agent) to provide a sulfoalkylated cellulose; and

(c) isolating the sulfoalkyl cellulose.

Alternatively, sulfoalkyl cellulose can be made by treating cellulose with an alkalizing agent(s) (e.g., aqueous sodium hydroxide) at the same time as treating the cellulose with a sulfoalkylating agent(s), or a combination of sulfoalkylating and carboxyalkylating agent(s).

In one embodiment, the method includes the following steps:

(a) treating cellulose with an alkaline solution of a sulfoalkylating agent (e.g., vinyl sulfonate) or with an alkaline solution of a sulfoalkylating agent and a carboxyalkylating agent (e.g., vinyl sulfonate and chloroacetic acid) to provide a sulfoalkylated cellulose; and

(b) isolating the sulfoalkyl cellulose.

In one embodiment, the cellulose is treated with alkali in a suspension comprising isopropanol. In one embodiment, the alkali includes sodium hydroxide.

In one embodiment, the sulfoalkylating agent is a haloethyl sulfonate, for example, chloroethyl sulfonate.

In one embodiment, the sulfoalkylating agent is a vinyl sulfonate, for example, sodium vinyl sulfonate.

In one embodiment, the sulfoalkylating agent is a 3-halo-2-hydroxypropyl sulfonate, for example, 3-chloro-2-hydroxypropyl sulfonate.

As noted above, the sulfoalkylated cellulose of the invention can be prepared by alkalizing cellulose to provide alkali cellulose, followed by etherifying the alkali cellulose with a sulfoalkylating agent. Alternatively, the sulfoalkylated cellulose of the invention can be prepared by alkalizing cellulose in the presence of vinyl sulfonate.

Alkali cellulose can be prepared in any one of a variety of ways. In a solvent-free method, fluff pulp (e.g., Retsch-milled fluff pulp) is wetted with a solution of aqueous sodium hydroxide (about 30-35% by weight sodium hydroxide) at low temperature (e.g., 0 to -5° C.). Alternatively, alkali cellulose can be prepared by a suspension method in which pulp is suspended in a water-miscible organic solvent (e.g., isopropanol) to provide a suspension having a consistency of from about 3 to about 10%. To the suspension is added an aqueous sodium hydroxide solution (30-35% by weight sodium hydroxide), or an aqueous sodium hydroxide solution containing vinyl sulfonate, at low temperature (e.g., 0 to -5° C.) with vigorous stirring so as to evenly distribute the alkali throughout the fibers. The resulting mixture is then ripened at low temperature for at least two hours, with the entire process being carried out under a nitrogen atmosphere.

The sulfoalkylated cellulose is prepared by reacting alkali cellulose with a sulfoalkylating agent (e.g., haloalkyl sulfonate or vinyl sulfonate). The alkali cellulose is reacted with the sulfoalkylating agent at a temperature from about 50° C. to about 80° C. under a nitrogen atmosphere for 3-9 hours with constant stirring. The sulfoalkylating agent can be added as a powder to a stirred suspension of the alkali cellulose in isopropanol.

In a representative method, haloalkyl sulfonates in powder form were added over a period of about 30 to 60 minutes to ripened alkali cellulose suspended in isopropanol under nitrogen while the temperature of the suspension was raised from ambient temperature to about 55° C. After the addition of the sulfoalkylating agents was complete, the mixture was heated at 55-60° C. for 3 to 9 hours. After cooling, the mixture was decanted or filtered, and the solids were washed sequentially with 75% aqueous isopropanol, acetic acid/isopropanol, and isopropanol, and dried.

In another representative embodiment, an aqueous solution of sodium hydroxide and sodium vinyl sulfonate were added over a 1 hour period to a pulp suspension in isopropanol. The mixture was kept at −5-0° C. for 90 minutes before slowly heating to 50-70° C. for 3-9 hours. A second sulfoalkylating agent (e.g., 3-chloro-2-hydroxypropyl sulfonate) was added and the mixture agitated with heating for 3-6 hours.

In one embodiment, the sulfoalkyl cellulose was obtained by dissolving the reaction product in water (e.g., to provide a 2-5% by weight solution) and then precipitating the cellulose from the solution by the addition of a non-solvent (e.g., isopropanol or acetone).

In one embodiment, the sulfoalkyl cellulose is obtained by treating alkali cellulose with an amount of two sulfoalkylating agents. This sulfoalkylated cellulose is obtained by sequential treatment with chloroethyl sulfonate or vinyl sulfonate followed by treatment with 3-chloro-2-hydroxypropyl sulfonate.

Cellulosic fibers are a starting material for preparing the sulfoalkyl cellulose useful in making the polymer networks of the invention. Although available from other sources, suitable cellulosic fibers are derived primarily from wood pulp. Suitable wood pulp fibers for use with the invention can be obtained from well-known chemical processes such as the kraft and sulfite processes, with or without subsequent bleaching, or crosslinking with suitable crosslinkers. Pulp fibers can also be processed by thermomechanical, chemithermomechanical methods, or combinations thereof. Caustic extractive pulp such as TRUCELL, commercially available from Weyerhaeuser Company, is also a suitable wood pulp fiber. A preferred pulp fiber is produced by chemical methods. Ground wood fibers, recycled or secondary wood pulp fibers, and bleached and unbleached wood pulp fibers can be used. Softwoods and hardwoods can be used. Details of the selection of wood pulp fibers are well-known to those skilled in the art. These fibers are commercially available from a number of companies, including Weyerhaeuser Company, the assignee of the present invention. For example, suitable cellulosic fibers produced from southern pine that are usable with the present invention are available from Weyerhaeuser Company under the designations CF416, NF405, PL416, FR416, and NB416. In one embodiment, the cellulosic fiber useful in making the polymer network of the invention is a southern pine fiber commercially available from Weyerhaeuser Company under the designation NB416. In other embodiments, the cellulosic fiber can be selected from among a northern softwood fiber, a eucalyptus fiber, a rye grass fiber, and a cotton fiber.

Cellulosic fibers having a wide range of degree of polymerization are suitable for making the sulfoalkyl cellulose. In one embodiment, the cellulosic fiber has a relatively high degree of polymerization, greater than about 1000, and in another embodiment, about 1500.

Sulfoalkyl celluloses, description of reagents used to make the sulfoalkyl celluloses, degree of sulfonate substitution, viscosity, and degree of carboxy substitution (as relevant) are summarized in Table 1. In Table 1, “VS” refers to vinyl sulfonate, “MCA” refers to monochloroacetic acid, “CES” refers to chloroethyl sulfonate, “CHPS” refers to 3-chloro-2-hydroxypropyl sulfonate, “Ratio” refers to the molar ratio of the alkylating agents, “NaOH/AGU” refers to the molar ratio of sodium hydroxide to anhydroglucose unit used in making the sulfoalkyl cellulose, “Agt/AGU” refers to the molar ratio of alkylating agent(s) to anhydroglucose unit used in making the sulfoalkyl cellulose, “DS_{COOH}” refers to the degree of carboxy substitution, and “DSS_{SO3Na}” refers to the degree of sulfonate substitution.

TABLE 1
Sulfoalkyl celluloses and properties.

| Sulfoalkyl | Alkylating | | | | | Viscosity | |
|---|---|---|---|---|---|---|---|
| cellulose | agent | Ratio | NaOH/AGU | Agt/AGU | DS_{COOH} | (cP) | DS_{SO3Na} |
| 11 | VS | — | 2.5 | 0.5 | 0.08 | 85 | 0.27 |
| 14 | VS/MCA | 1.0:1.0 | 2.5 | 0.5 | 0.21 | 65 | 0.18 |
| 15 | VS/MCA | 1.0:1.0 | 2.5 | 0.3 | 0.04 | 100 | 0.081 |
| 16 | VS/MCA | 1.0:1.0 | 2.5 | 0.8 | 0.24 | 65 | 0.22 |
| 17 | VS/CES | 1.0:1.0 | 2.5 | 0.5 | — | 32 | 0.286 |
| 18 | VS/CES | 1.0:1.0 | 2.5 | 1.0 | — | 50 | 0.55 |
| 19 | VS/CES | 1.0:1.0 | 2.5 | 2.0 | — | 65 | 0.93 |
| 20 | VS/CES | 1.0:1.0 | 2.5 | 1.0 | — | 41 | 0.51 |
| 23 | VS/CES | 1.0:1.0 | 1.25 | 1.0 | — | 100 | 0.53 |
| 26 | VS | — | 0.65 | 1.0 | — | 85 | 0.27 |
| 35 | VS/MCA | 1.0:1.0 | 1.5 | 1.0 | 0.27 | 100 | 0.15 |
| 37 | VS/MCA | 1.0:1.0 | 1.5 | 1.5 | 0.47 | 125 | 0.13 |
| 38 | VS/MCA | 1.0:1.0 | 1.5 | 2.0 | 0.51 | 275 | 0.18 |
| 39 | VS/MCA | 1.0:2.0 | 1.5 | 1.5 | 0.59 | 112.5 | 0.09 |
| 42 | VS/MCA | 1.0:1.0 | 1.5 | 1.0 | — | 225 | 0.13 |
| 44 | VS/MCA | 1.0:1.0 | 1.5 | 1.0 | 0.32 | 65 | 0.22 |
| 46 | CES/MCA | 1.0:1.0 | 1.5 | 1.0 | — | 140 | 0.12 |
| 48 | VS/MCA | 1.0:0.5 | 1.5 | 0.75 | 0.18 | 125 | 0.22 |
| 49 | VS/MCA | 1.0:1.5 | 1.5 | 1.3 | 0.43 | 100 | 0.17 |
| 51 | VS/MCA | 1.0:2.0 | 1.5 | 1.5 | 0.57 | 100 | 0.14 |
| 53 | VS/MCA | 1.0:2.5 | 1.5 | 1.75 | 0.7 | 125 | 0.12 |
| 56 | VS | — | 1.5 | 1.0 | — | 65 | 0.52 |
| 58 | VS | — | 1.5 | 1.5 | — | 250 | 0.42 |
| 60 | VS | — | 1.5 | 2.0 | — | 22.5 | |
| 62 | VS | — | 2.5 | 1.5 | — | 50 | 0.71 |
| 64 | VS + CHPS | 1.0:1.0 | 1.5 | 2.0 | — | 41 | 0.745 |
| 68 | VS/CHPS | 1.0:0.5 | 1.5 | 1.5 | — | 85 | 0.83 |
| 72 | VS + CHPS | 0.5:1.0 | 2.5 | 1.5 | — | 50 | 0.54 |
| 76 | VS | — | 2.5 | 0.5 | — | — | 0.28 |
| 78 | VS | — | 2.5 | 0.5 | — | — | 0.31 |
| 80 | VS | — | 3.0 | 0.5 | — | — | 0.32 |

As noted above, the sulfoalkyl cellulose polymer network includes two types of polymers: a sulfoalkyl cellulose and a synthetic water-soluble polymer.

As used herein, the term “synthetic” refers to a polymer that is made by chemical synthesis (e.g., polyacrylic acid or polyacrylamide) and is not a naturally-occurring polymer (e.g., cellulose). In one embodiment, the synthetic water-soluble polymer is a synthetic polymer having carboxylic acid substituents. In one embodiment, the synthetic water-soluble polymer is a synthetic polymer having carboxylic acid derivative substituents. In one embodiment, the synthetic water-soluble polymer is a synthetic polymer having carboxylic acid substituents and carboxylic acid derivative substituents.

The term “carboxylic acid substituent” refers to a free acid substituent having the formula —CO₂H; a carboxylate substituent having the formula —CO₂⁻; or a carboxylate salt substituent having the formula —CO₂M, where M is a cationic species such as a metal ion (e.g., sodium or potassium). The term “carboxylic acid derivative substituent” refers to a substituent having the formula —COXR. The carboxylic acid derivative substituent can be an amide (i.e., —CONH₂, —CONHR₁, or —CONR²₂, where R¹ and R² are alkyl groups). Other suitable carboxylic acid derivative substituents include ester substituents. In one embodiment, the carboxylic acid derivative substituent is an amide.

Representative polymers having carboxylic acid substituents include polyacrylic acid polymers, polymaleic acid polymers, polyaspartic acid polymers, copolymers of acrylic acid and acrylamide, copolymers of acrylic acid and maleic acid, copolymers of maleic acid and itaconic acid, partially-hydrolyzed polyacrylamide polymers, and mixtures thereof. In one embodiment, the water soluble polymer is a polyacrylic acid. Suitable polyacrylic acid polymers include polyacrylic acids having a variety of molecular weights. Exemplary polyacrylic acid polymers have the following molecular weights: 450,000; 750,000; 1,250,000, 3,000,000; and 4,000,000.

Representative polymers having carboxylic acid derivative substituents include polyacrylamide polymers. In one embodiment, the water-soluble polymer is a polyacrylamide. Suitable polyacrylamide polymers include polyacrylamides having a variety of molecular weights. Exemplary polyacrylamide polymers have the following molecular weight ranges: 5,000,000 to 6,000,000, and 11,000,000 to 14,000,000.

Other representative water-soluble polymers include polyvinyl alcohol (PVA), polyoxyethylene (PEG), polyoxypropylene, and a polyoxyethylene/polyoxypropylene block copolymer.

The composition can be made from mixtures of water-soluble polymers.

In one embodiment, the water-soluble polymer is a polyacrylic acid. In one embodiment, the water-soluble polymer is a polyacrylamide.

As noted above, the polymer network is obtained by reacting a sulfoalkyl cellulose and a water-soluble polymer with a crosslinking agent.

Suitable crosslinking agents include crosslinking agents that are reactive toward carboxylic acid groups. Representative organic crosslinking agents that are reactive toward carboxylic acid groups include diols and polyols, diamines and polyamines, diepoxides and polyepoxides, polyoxazoline functionalized polymers, and aminols having one or more amino groups and one or more hydroxy groups. Representative inorganic crosslinking agents that are reactive toward carboxylic acid groups include polyvalent cations and polycationic polymers. Exemplary inorganic crosslinking agents include aluminum chloride, aluminum sulfate, and ammonium zirconium carbonate with or without carboxylic acid ligands such as succinic acid (dicarboxylic acid), citric acid (tricarboxylic acid), butane tetracarboxylic acid (tetracarboxylic acid). Water soluble salts of trivalent iron and divalent zinc and copper can be used as crosslinking agents. Clay materials such as Kaolinite and Montmorrillonite can also be used for crosslinking polycarboxylated polymers. Titanium alkoxides commercially available from DuPont under the designation TYZOR can be used to form covalent bonds with polymer carboxyl and/or hydroxyl groups.

Mixtures of crosslinking agents can be used.

Representative diol crosslinking agents include 1,4-butanediol and 1,6-hexanediol.

Representative diamine and polyamine crosslinking agents include polyether diamines, such as polyoxypropylenediamine, and polyalkylene polyamines. Suitable polyether diamines and polyether polyamines are commercially available from Huntsman Corp., Houston, Tex., under the designation JEFFAMINE. Representative diamines and polyamines (e.g., tri-, tetra-, and pentaamines) include JEFFAMINE D-230 (molecular weight 230), JEFFAMINE D-400 (molecular weight 400), and JEFFAMIE D-2000 (molecular weight 2000); JEFFAMINE XTJ-510 (D-4000) (molecular weight 4000), JEFFAMINE XTJ-50 (ED-600) (molecular weight 600), JEFFAMINE XTJ-501 (ED-900) (molecular weight 900), and JEFFAMINE XTJ-502 (ED-2003) (molecular weight 2000); JEFFAMINE XTJ-504 (EDR-148) (molecular weight 148); JEFFAMINE HK-511 (molecular weight 225); and ethylenediamine, diethylenetriamine, triethylenetetraamine, and tetraethylenepentaamine.

Representative diepoxide crosslinking agents include vinylcyclohexene dioxide, butadiene dioxide, and diglycidyl ethers such as polyethylene glycol (400) diglycidyl ether and ethylene glycol diglycidyl ether.

Representative polyoxazoline functionalized polymers include EPOCROS WS-500 manufactured by Nippon Shokubai.

Representative aminol crosslinking agents include triethanolamine.

Representative polycationic polymers include polyethylenimine and polyamido epichlorohydrin resins such as KYMENE 557H manufactured by Hercules, Inc.

Suitable crosslinking agents include crosslinking agents that are reactive toward the synthetic water-soluble polymer functional groups and/or the sulfoalkyl cellulose hydroxyl groups. Representative crosslinking agents that are reactive toward the cellulose hydroxyl groups include aldehyde, dialdehyde, dialdehyde sodium bisulfite addition product, dihalide, diene, diepoxide, haloepoxide, dicarboxylic acid, and polycarboxylic acid crosslinking agents. Mixtures of crosslinking agents can also be used.

Representative aldehyde crosslinking agents include formaldehyde.

Representative dialdehyde crosslinking agents include glyoxal, glutaraldehyde, and dialdehyde sodium bisulfite addition products.

Representative dihalide crosslinking agents include 1,3-dichloro-2-hydroxypropane.

Representative diene crosslinking agents include divinyl ethers and divinyl sulfone.

Representative diepoxide crosslinking agents include vinylcyclohexene dioxide, butadiene dioxide, and diglycidyl ethers such as polyethylene glycol diglycidyl ether and ethylene glycol diglycidyl ether.

Representative haloepoxide crosslinking agents include epichlorohydrin.

Representative carboxylic acid crosslinking agents including di- and polycarboxylic acids. U.S. Pat. Nos. 5,137,537, 5,183,707, and 5,190,563, describe the use of C2-C9 polycarboxylic acids that contain at least three carboxyl groups (e.g., citric acid and oxydisuccinic acid) as crosslinking agents. Suitable polycarboxylic acid crosslinking agents include citric acid, tartaric acid, malic acid, succinic acid, glutaric acid, citraconic acid, itaconic acid, tartrate monosuccinic acid, maleic acid, 1,2,3-propane tricarboxylic acid, 1,2,3,4-butanetetracarboxylic acid, all-cis-cyclopentane tetracarboxylic acid, tetrahydrofuran tetracarboxylic acid, 1,2,4,5-benzenetetracarboxylic acid, and benzenehexacarboxylic acid.

Carboxyalkyl celluloses and carboxylated synthetic polymers may be crosslinking with diamines and polyamines. Depending on the diamine or polyamine, the polymers may be crosslinked through diamide crosslinks or amide/ionic crosslinks. A mixture of a first carboxylated polymer having a plurality of carboxyl groups and a second carboxylated polymer having a plurality of carboxyl groups can be treated with a triazine crosslinking activator (e.g., 2,4,6-trichloro-1,3,5-triazine, also known as cyanuric chloride, and 2-chloro-4,6-dimethoxy-1,3,5-triazine) to provide a mixture of first and second activated carboxylated polymers. In one embodiment, the mixture of activated carboxylated polymers is reacted with a diamine or polyamine having two amino groups (e.g., primary and secondary amino groups) reactive toward activated carboxyl groups of the first and second activated carboxylated polymers to form a plurality of diamide crosslinks to provide a crosslinked carboxylated polymer. In another embodiment, the mixture of activated carboxylated polymers is reacted with a diamine or polyamine having one amino group that is reactive toward the activated carboxyl groups of the first and second activated carboxylated polymers to form a plurality of amide bonds, and a second amino group (e.g., tertiary and quaternary amino groups) that is not covalently reactive toward the activated carboxyl groups of the first and second activated carboxylated polymers and forms a plurality of ionic bonds with carboxyl groups, thereby effectively crosslinking the polymers to provide a crosslinked carboxylated polymer. The term “ionic crosslink” refers to a crosslink that includes an amide bond and an ionic bond or association between an amino group and a carboxyl group. An ionic crosslink is formed by reaction of a first activated carboxyl group with a diamine or polyamine to provide a first amide, the resulting amide having a second amino group that is ionically reactive or associative toward a second carboxyl group.

It will be appreciated that mixtures and/or blends of crosslinking agents can also be used.

Crosslinking catalysts can be used to accelerate the crosslinking reaction. Suitable catalysts include acidic salts, such as ammonium chloride, ammonium sulfate, aluminum chloride, magnesium chloride, and alkali metal salts of phosphorous-containing acids.

The amount of crosslinking agent applied to the sulfoalkyl cellulose and synthetic water-soluble polymers can vary depending on the desired absorption characteristics. The amount of crosslinking agent applied will depend on the particular crosslinking agent and is suitably in the range of from about 0.01 to about 8.0 percent by weight based on the total weight of the sulfoalkyl cellulose. In one embodiment, the amount of crosslinking agent applied is in the range from about 0.50 to about 5.0 percent by weight based on the total weight of the sulfoalkyl cellulose. In one embodiment, the amount of crosslinking agent applied is in the range from about 1.0 to about 2.0 percent by weight based on the total weight of the sulfoalkyl cellulose.

The sulfoalkyl cellulose polymer network of the invention has a Free Swell Capacity of at least about 10 g/g. In one embodiment, the network has a Free Swell Capacity of from about 10 g/g to about 90 g/g. Free Swell Capacity was determined by the method described in Example 1.

The polymer network of the invention has a Centrifuge Capacity of at least about 2 g/g. In one embodiment, the network has a Centrifuge Capacity of from about 2 g/g to about 60 g/g. Centrifuge Capacity was determined by the method described in Example 1.

The polymer network of the invention has an Absorbency Under Load (AUL) value of at least about 5 g/g. In one embodiment, the network has an Absorbency Under Load value of from about 5 g/g to about 40 g/g. Absorbency Under Load value was determined by the method described in Example 2.

In another aspect, of the invention, a method for making a sulfoalkyl cellulose polymer network having superabsorbent properties is provided. In the method, a sulfoalkyl cellulose and a synthetic water-soluble polymer are reacted with a crosslinking agent. The crosslinking agent reacts with at least one of the sulfoalkyl cellulose or synthetic water-soluble polymer.

In one embodiment, the method comprises treating a sulfoalkyl cellulose and a synthetic water-soluble polymer with a crosslinking agent to provide a reaction mixture, and crosslinking the reaction mixture to provide the composition.

In another embodiment, the method comprises combining a sulfoalkyl cellulose, a synthetic water-soluble polymer, and a crosslinking agent in an aqueous solution to provide a reaction mixture; precipitating the reaction mixture by addition of a water-miscible solvent to provide a precipitated mixture; collecting the precipitated mixture; and crosslinking the precipitated mixture to provide the polymer network.

In embodiments using certain metal ions as the crosslinking agent, combining a solution of a sulfoalkyl cellulose with the metal ion (e.g., aluminum sulfate) results in precipitation of a crosslinked product at or near room temperature (i.e., about 25° C.).

In other embodiments, crosslinking can be achieved by heating at a temperature and for a period of time sufficient to effect crosslinking. Crosslinking can be achieved by heating at a temperature of about 50 to 150° C. for about 5 to 60 minutes. Crosslinking can occur during precipitation of the polymer mixture or during evaporation of the precipitated mixture to dryness.

In one embodiment, the method further includes combining the sulfoalkyl cellulose, the synthetic water-soluble polymer, and the crosslinking agent with a second crosslinking agent. The second crosslinking agent is different from the crosslinking agent initially combined with the sulfoalkyl cellulose and the synthetic water-soluble polymer.

Thus, in another aspect, the invention provides a polymer network obtainable from the reaction of a sulfoalkyl cellulose and a synthetic water-soluble polymer with two crosslinking agents. Each crosslinking agent reacts with at least one of the sulfoalkyl cellulose or synthetic water-soluble polymer.

The second crosslinking agent can be any one of those described above including aldehyde, dialdehyde, dihalide, diene, diepoxide, haloepoxide, dicarboxylic acid, polycarboxylic acid, diol, diamine, aminol, inorganic cationic compound, and polycationic polymer crosslinking agents.

The second crosslinking agent is added in an amount from about 2 to about 20 mole percent based on the amount of the synthetic water-soluble polymer. In one embodiment, the second crosslinking agent is added in an amount from about 4 to about 16 mole percent based on the amount of the synthetic water-soluble polymer. In one embodiment, the second crosslinking agent is added in an amount from about 6 to about 10 mole percent based on the amount of the synthetic water-soluble polymer.

Tables 2-6 summarize representative sulfoalkyl cellulose polymer networks of the invention; the sulfoalkyl celluloses, synthetic water-soluble polymers, and crosslinking agents from which they are made; reaction times and temperatures for making the polymer networks; form of the polymer networks; and Free Swell and Centrifuge Capacities, and Absorbency Under Load values. In the tables, “form” refers to the method for isolating the polymer network; the term “ppt” refers to polymer networks isolated by precipitation from water solution using a water-miscible non-solvent; the term “film” refers to polymer networks isolated by evaporation of the water solution; the term “PAA” refers to a polyacrylic acid; the term “PAM1” refers to a polyacrylamide commercially available from Polysciences having a molecular weight of 5-6 million; and the term “PAM2” refers to a polyacrylamide commercially available from JRM Chemical having a molecular weight of 11-14 million. In the tables, synthetic polymer amount refers to the percent by weight synthetic polymer based on the weight of sulfoalkyl cellulose; and crosslinking agent amount (XL%) for mixed networks with polyacrylic acid refers to the percent by weight crosslinking agent applied based on the total weight of all polymers. For mixed networks of sulfoalkylated celluloses with polyacrylamides, the amount of crosslinking agent (XL%) is mole percent based on polyacrylamide.

Table 2 summarizes representative sulfoalkyl cellulose polymer networks made from sulfoalkyl cellulose combined with polyacrylamide or polyacrylic acid crosslinked with divinyl sulfone (Entry 1) or glutaraldehyde (Entries 2-26). The sulfoalkyl cellulose was made by sulfoalkylating with vinyl sulfonate.

TABLE 2
Representative sulfoalkyl cellulose polymer networks and properties.

| | Synthetic | Polymer | | | | Free | | |
|---|---|---|---|---|---|---|---|---|
| Sulfoalkyl | polymer | Network | XL | Temp | Time | Swell | Centrifuge | AUL |
| Cellulose | (%) | Form | (%) | (° C.) | (min) | (g/g) | (g/g) | (g/g) |
| 11 | PAA (9) | ppt | 1 (12.5) | — | — | 24.25 | 6.75 | — |
| 11 | PAA (10) | ppt | 2 (4.3) | — | — | 41.44 | 20.49 | 14.14 |
| 11 | PAM2 (10) | ppt | 2 (0.22) | — | — | 42.83 | 23.88 | 14.68 |
| 11 | PAM2 (10) | ppt | 2 (0.22) | 150 | 15 | 46.06 | 28.26 | 18.67 |
| 26 | PAA (9.8) | ppt | 2 (4.3) | — | — | 31.99 | 9.06 | 20.63 |
| 56 | PAA (10) | film | 2 (4.3) | — | — | 22.75 | 9.93 | 5.62 |
| 56 | PAM1 (10) | ppt | 2 (0.22) | — | — | 42.57 | 19.34 | — |
| 56 | PAM1 (10) | ppt | 2 (0.22) | 150 | 15 | 31.78 | 17.41 | — |
| 56 | PAM1 (10) | ppt | 2 (0.22) | 150 | 30 | 35.84 | 18.88 | — |
| 58 | PAA (10) | film | 2 (4.3) | — | — | 19.95 | 5.49 | 10.37 |
| 58 | PAM1 (10) | ppt | 2 (0.22) | — | — | 45.20 | 21.36 | — |
| 58 | PAM1 (10) | ppt | 2 (0.22) | 150 | 15 | 37.89 | 16.87 | — |
| 58 | PAM1 (10) | ppt | 2 (0.22) | 150 | 30 | 37.80 | 15.06 | — |
| 62 | PAA (10) | film | 2 (4.3) | — | — | 32.84 | 17.14 | 12.39 |
| 62 | PAM1 (10) | ppt | 2 (0.22) | — | — | 38.74 | 17.70 | — |
| 62 | PAM1 (10) | ppt | 2 (0.22) | 150 | 15 | 34.41 | 18.59 | — |
| 62 | PAM1 (10) | ppt | 2 (0.22) | 150 | 30 | 34.39 | 18.36 | — |
| 76 | PAM1 (10) | ppt | 2 (0.22) | — | — | 31.01 | 11.59 | — |
| 76 | PAM1 (10) | ppt | 2 (0.22) | 150 | 15 | 32.11 | 11.14 | — |
| 76 | PAM1 (10) | ppt | 2 (0.22) | 150 | 30 | 30.65 | 10.02 | — |
| 78 | PAM1 (10) | ppt | 2 (0.22) | — | — | 25.84 | 10.93 | — |
| 78 | PAM1 (10) | ppt | 2 (0.22) | 150 | 15 | 25.26 | 11.93 | — |
| 78 | PAM1 (10) | ppt | 2 (0.22) | 150 | 30 | 27.25 | 12.36 | — |
| 80 | PAM1 (10) | ppt | 2 (0.22) | — | — | 28.83 | 13.71 | — |
| 80 | PAM1 (10) | ppt | 2 (0.22) | 150 | 15 | 30.04 | 14.46 | — |
| 80 | PAM1 (10) | ppt | 2 (0.22) | 150 | 30 | 31.02 | 14.71 | — |

Table 3 summarizes representative sulfoalkyl cellulose polymer networks made from sulfoalkyl cellulose combined with polyacrylamide or polyacrylic acid crosslinked with glutaraldehyde. The sulfoalkyl cellulose was made by sulfoalkylating with vinyl sulfonate and chloroethyl sulfonate.

TABLE 3
Representative sulfoalkyl cellulose polymer networks and properties.

| Sulfoalkyl | Synthetic | | | Temp | Time | Free Swell | Centrifuge | AUL |
|---|---|---|---|---|---|---|---|---|
| Cellulose | polymer (%) | Form | XL (%) | (° C.) | (min) | (g/g) | (g/g) | (g/g) |
| 17 | PAM2 (10) | ppt | 0.22 | — | — | 33.49 | 19.09 | 11.83 |
| 17 | PAM2 (10) | ppt | 0.22 | 150 | 15 | 38.51 | 22.86 | 16.33 |
| 19 | PAM1 (10) | ppt | 0.22 | — | — | 18.13 | 7.53 | — |
| 19 | PAM1 (10) | ppt | 0.22 | 150 | 15 | 16.44 | 8.09 | — |
| 20 | PAM1 (10) | ppt | 0.22 | — | — | 24.64 | 9.92 | 11.80 |
| 20 | PAM1 (10) | ppt | 0.22 | 150 | 15 | 24.99 | 15.77 | 16.40 |
| 23 | PAM2 (10) | ppt | 0.22 | — | — | 34.72 | 21.30 | 22.00 |
| 23 | PAM2 (10) | ppt | 0.22 | 150 | 15 | 51.14 | 36.71 | 26.27 |
| 23 | PAA (9.8) | ppt | 4.3 | — | — | 44.49 | 20.48 | 15.00 |
| 23 | PAA (10) | film | 4.3 | — | — | 12.40 | 2.38 | 5.27 |

Table 4 summarizes representative sulfoalkyl cellulose polymer networks made from sulfoalkyl cellulose combined with polyacrylic acid crosslinked with glutaraldehyde. The sulfoalkyl cellulose was made by sulfoalkylating with vinyl sulfonate and 3-chloro-2-hydroxypropyl sulfonate.

TABLE 4
Representative sulfoalkyl cellulose polymer
networks and properties.

| | Syn- | | | | | | | |
|---|---|---|---|---|---|---|---|---|
| | thetic | | | | | Free | Centri- | |
| Sulfoalkyl | polymer | | XL | Temp | Time | Swell | fuge | AUL |
| Cellulose | (%) | Form | (%) | (° C.) | (min) | (g/g) | (g/g) | (g/g) |
| 64 | PAA | film | 4.3 | — | — | 24.13 | 12.36 | 6.37 |
| | (10) | | | | | | | |
| 68 | PAA | film | 4.3 | — | — | 21.76 | 8.53 | 7.17 |
| | (10) | | | | | | | |
| 72 | PAA | film | 4.3 | — | — | 25.98 | 12.07 | 9.55 |
| | (10) | | | | | | | |

Table 5 summarize a representative sulfoalkyl cellulose polymer network made from sulfoalkyl cellulose combined with polyacrylic acid crosslinked with glutaraldehyde. The sulfoalkyl cellulose was made by carboxyalkylating with chloroacetic acid and sulfoalkylating with chloroethyl sulfonate.

TABLE 5
Representative sulfoalkyl cellulose polymer
network and properties.

| | Syn- | | | | | | | |
|---|---|---|---|---|---|---|---|---|
| | thetic | | | | | Free | Cen- | |
| Sulfoalkyl | polymer | | XL | Temp | Time | Swell | trifuge | AUL |
| Cellulose | (%) | Form | (%) | (° C.) | (min) | (g/g) | (g/g) | (g/g) |
| 46 | PAA/ | film | 4.3 | — | — | 35.06 | 19.24 | 13.82 |
| | 10% | | | | | | | |

Table 6 summarizes representative sulfoalkyl cellulose polymer networks made from sulfoalkyl cellulose combined with polyacrylamide or polyacrylic acid and crosslinked with glutaraldehyde. The sulfoalkyl cellulose was made by carboxyalkylating with chloroacetic acid and sulfoalkylating with vinyl sulfonate.

TABLE 6
Representative sulfoalkyl cellulose polymer
networks and properties.

| Sulfo- | | | | | | | Cen- | |
|---|---|---|---|---|---|---|---|---|
| alkyl | Synthetic | | | | | Free | tri- | |
| Cellu- | polymer | | XL | Temp | Time | Swell | fuge | AUL |
| lose | (%) | Form | (%) | (° C.) | (min) | (g/g) | (g/g) | (g/g) |
| 14 | PAA (9.8) | ppt | 4.3 | — | — | 33.10 | 17.93 | 14.92 |
| 14 | PAM2 (10) | ppt | 0.22 | — | — | 30.17 | 11.13 | 16.72 |
| 14 | PAM2 (10) | ppt | 0.22 | 150 | 15 | 34.24 | 9.76 | 23.60 |
| 15 | PAA (9.8) | ppt | 4.3 | — | — | 23.23 | 4.42 | 13.01 |
| 15 | PAM2 (10) | ppt | 0.22 | — | — | 34.80 | 20.66 | 15.29 |
| 15 | PAM2 (10) | ppt | 0.22 | 150 | 15 | 35.89 | 22.88 | 17.08 |
| 16 | PAA (9.8) | ppt | 4.3 | — | — | 34.42 | 16.47 | 12.13 |
| 16 | PAM2 (10) | ppt | 0.22 | — | — | 24.42 | 11.12 | 12.35 |
| 16 | PAM2 (10) | ppt | 0.22 | 150 | 15 | 38.28 | 23.35 | 16.39 |
| 35 | PAA (10) | film | 4.3 | — | — | 34.76 | 19.94 | 13.64 |
| 35 | PAM1 (10) | ppt | 0.22 | — | — | 38.06 | 19.98 | 15.50 |
| 35 | PAM1 (10) | ppt | 0.22 | 150 | 15 | 37.54 | 22.31 | 21.30 |
| 35 | PAM1 (10) | ppt | 0.22 | 150 | 30 | 37.66 | 22.34 | 16.90 |
| 37 | PAA (10) | film | 4.3 | — | — | 41.94 | 25.11 | 14.75 |
| 37 | PAM1 (10) | ppt | 0.22 | — | — | 41.32 | 21.34 | 16.30 |
| 37 | PAM1 (10) | ppt | 0.22 | 150 | 15 | 39.63 | 25.41 | 18.60 |
| 37 | PAM1 (10) | ppt | 0.22 | 150 | 30 | 39.17 | 25.04 | 19.40 |
| 38 | PAA (10) | film | 4.3 | — | — | 44.29 | 25.52 | 15.07 |
| 38 | PAM1 (10) | ppt | 0.22 | — | — | 49.58 | 33.31 | 20.10 |
| 38 | PAM1 (10) | ppt | 0.22 | 150 | 15 | 50.29 | 33.32 | 20.02 |
| 38 | PAM1 (10) | ppt | 0.22 | 150 | 30 | 49.83 | 26.17 | 21.69 |
| 39 | PAA (10) | film | 4.3 | — | — | — | — | — |
| 39 | PAM1 (10) | ppt | 0.22 | — | — | 44.27 | 24.36 | 17.50 |
| 39 | PAM1 (10) | ppt | 0.22 | 150 | 15 | 41.66 | 24.21 | 19.20 |
| 39 | PAM1 (10) | ppt | 0.22 | 150 | 30 | 42.24 | 25.60 | 18.60 |
| 42 | PAA (10) | film | 4.3 | — | — | — | — | — |
| 42 | PAM1 (10) | ppt | 0.22 | — | — | 46.38 | 24.86 | 23.06 |
| 42 | PAM1 (10) | ppt | 0.22 | 150 | 15 | 44.13 | 22.46 | 22.14 |
| 42 | PAM1 (10) | ppt | 0.22 | 150 | 30 | 38.32 | 16.75 | 23.24 |
| 44 | PAA (10) | film | 4.3 | — | — | 74.84 | 45.83 | 21.84 |
| 44 | PAA (10) | film | 4.3 | — | — | 37.82 | 21.39 | 13.86 |
| 44 | PAA (10) | film | 4.3 | — | — | 35.85 | 20.25 | 11.71 |
| 44 | PAM1 (10) | ppt | 0.22 | — | — | 43.35 | 20.05 | 17.13 |
| 44 | PAM1 (10) | ppt | 0.22 | 150 | 15 | 42.66 | 25.62 | 19.93 |
| 44 | PAM1 (10) | ppt | 0.22 | 150 | 30 | 46.66 | 28.94 | 21.04 |
| 48 | PAA (10) | film | 4.3 | — | — | 23.31 | 8.63 | 10.42 |
| 48 | PAM1 (10) | ppt | 0.22 | — | — | 59.86 | 36.23 | 19.13 |
| 48 | PAM1 (10) | ppt | 0.22 | 150 | 15 | 52.35 | 34.39 | 20.12 |
| 48 | PAM1 (10) | ppt | 0.22 | 150 | 30 | 49.49 | 28.50 | 19.94 |
| 49 | PAA (10) | film | 4.3 | — | — | 32.91 | 16.77 | 12.85 |
| 49 | PAM1 (10) | ppt | 0.22 | — | — | 30.54 | 14.34 | 17.87 |
| 49 | PAM1 (10) | ppt | 0.22 | 150 | 15 | 28.84 | 17.63 | 19.39 |
| 49 | PAM1 (10) | ppt | 0.22 | 150 | 30 | 41.80 | 28.55 | 19.20 |
| 51 | PAA (10) | film | 4.3 | — | — | 46.73 | 22.87 | 15.28 |
| 51 | PAM1 (10) | ppt | 0.22 | — | — | 33.31 | 16.69 | 17.43 |
| 51 | PAM1 (10) | ppt | 0.22 | 150 | 15 | 35.18 | 21.81 | 18.00 |
| 51 | PAM1 (10) | ppt | 0.22 | 150 | 30 | 45.15 | 30.26 | 18.67 |
| 53 | PAA (10) | film | 4.3 | — | — | 52.41 | 18.47 | 16.34 |
| 53 | PAM1 (10) | ppt | 0.22 | — | — | 31.29 | 15.60 | 17.07 |
| 53 | PAM1 (10) | ppt | 0.22 | 150 | 15 | 30.44 | 20.04 | 17.79 |
| 53 | PAM1 (10) | ppt | 0.22 | 150 | 30 | 42.85 | 29.08 | 22.26 |

In another aspect, the invention provides absorbent products that include the sulfoalkyl cellulose polymer network described above. The sulfoalkyl cellulose polymer network can be incorporated into a personal care absorbent product. The sulfoalkyl cellulose polymer network can be formed into a composite for incorporation into a personal care absorbent product. Composites can be formed from the sulfoalkyl cellulose polymer network alone or by combining the sulfoalkyl cellulose polymer network with other materials, including fibrous materials, binder materials, other absorbent materials, other materials commonly employed in personal care absorbent products. Suitable fibrous materials include synthetic fibers, such as polyester, polypropylene, and bicomponent binding fibers; and cellulosic fibers, such as fluff pulp fibers, crosslinked cellulosic fibers, cotton fibers, and CTMP fibers. Suitable absorbent materials include natural absorbents, such as sphagnum moss, and synthetic superabsorbents, such as polyacrylates (e.g., SAPs).

Absorbent composites derived from or that include the sulfoalkyl cellulose polymer network can be advantageously incorporated into a variety of absorbent articles such as diapers including disposable diapers and training pants; feminine care products including sanitary napkins, and pant liners; adult incontinence products; toweling; surgical and dental sponges; bandages; food tray pads; and the like. Thus, in another aspect, the present invention provides absorbent composites, constructs, and absorbent articlaes that include the sulfoalkyl cellulose polymer network.

The sulfoalkyl cellulose polymer network can be incorporated as an absorbent core or storage layer into a personal care absorbent product such as a diaper. The composite can be used alone or combined with one or more other layers, such as acquisition and/or distribution layers, to provide useful absorbent constructs.

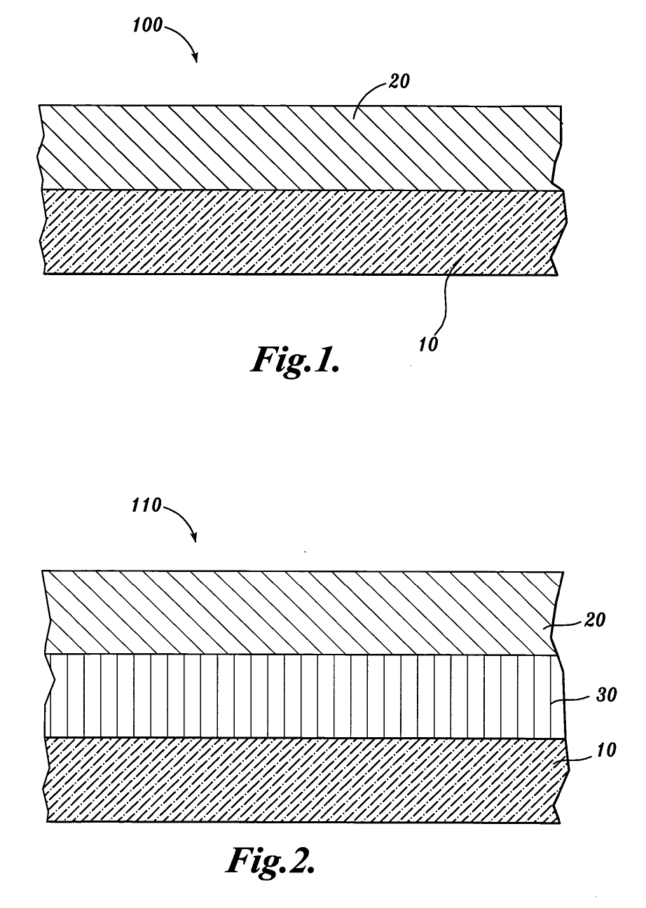

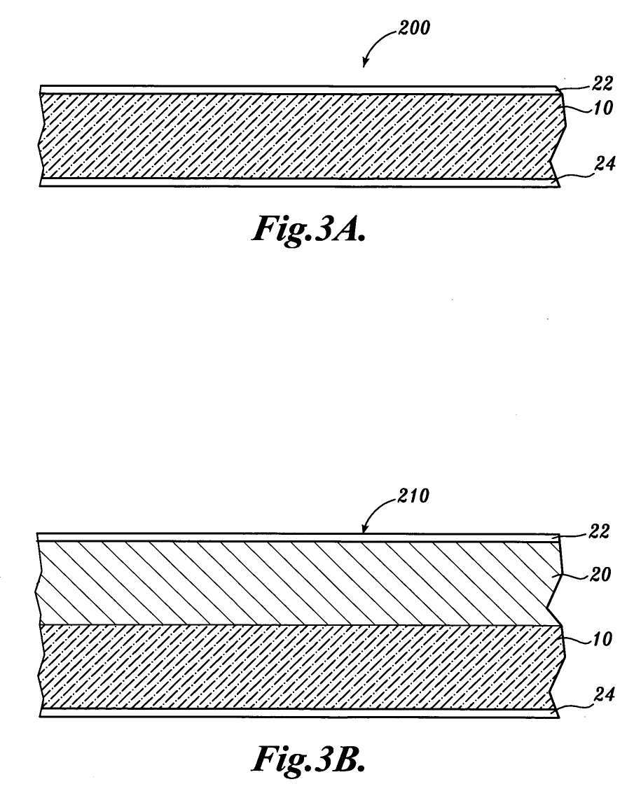

Representative absorbent constructs incorporating an absorbent composite that includes the sulfoalkyl cellulose polymer network are shown in FIGS. 1 and 2. Referring to FIG. 1, construct 100 includes composite 10 (i.e., a composite that includes a sulfoalkyl cellulose polymer network) employed as a storage layer in combination with an upper acquisition layer 20.

In addition to the construct noted above that includes the combination of absorbent composite and acquisition layer, further constructs can include a distribution layer intermediate the acquisition layer and composite. FIG. 2 illustrates construct 110 having intermediate layer 30 (e.g., distribution layer) interposed between acquisition layer 20 and composite 10.

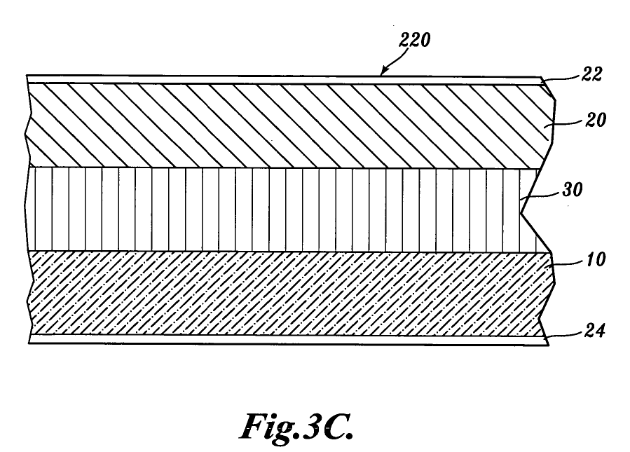

Composite 10 and constructs 100 and 110 can be incorporated into absorbent articles. Generally, absorbent articles 200, 210, and 220 shown in FIGS. 3A-C, include liquid pervious facing sheet 22, liquid impervious backing sheet 24, and a composite 10, construct 100, construct 110, respectively. In such absorbent articles, the facing sheet can be joined to the backing sheet.

It will be appreciated that other absorbent products can be designed incorporating the sulfoalkyl cellulose polymer network and composites that include the polymer network.

The following examples are provided for the purpose of illustrating, not limiting, the present invention.

### EXAMPLES

### Example 1

### Method for Determining Free Swell Capacity and Centrifuge Capacity

In this example, a method for determining free swell capacity (g/g) and centrifuge capacity (g/g) is described.

The materials, procedure, and calculations to determine free swell capacity (g/g) and centrifuge capacity (g/g) were as follows.

Test Materials:

Japanese pre-made empty tea bags (available from Drugstore.com, IN PURSUIT OF TEA polyester tea bags 93 mm×70 mm with fold-over flap. (http:www.mesh.ne.jp/tokiwa/).

Balance (4 decimal place accuracy, 0.0001 g for air-dried superabsorbent polymer (AD SAP) and tea bag weights).

Timer.

1% Saline.

Drip rack with clips (NLM 211)

Lab centrifuge (NLM 211, Spin-X spin extractor, model 776S, 3,300 RPM, 120v).

Test Procedure:

1. Determine solids content of AD SAP.

2. Pre-weigh tea bags to nearest 0.0001 g and record.

3. Accurately weigh 0.2025 g+/−0.0025 g of test material (SAP), record and place into pre-weighed tea bag (air-dried (AD) bag weight). (AD SAP weight+AD bag weight=total dry weight).

4. Fold tea bag edge over closing bag.

5. Fill a container (at least 3 inches deep) with at least 2 inches with 1% saline.

6. Hold tea bag (with test sample) flat and shake to distribute test material evenly through bag.

7. Lay tea bag onto surface of saline and start timer.

8. Soak bags for specified time (e.g., 30 minutes).

9. Remove tea bags carefully, being careful not to spill any contents from bags, hang from a clip on drip rack for 3 minutes.

10. Carefully remove each bag, weigh, and record (drip weight).

11. Place tea bags onto centrifuge walls, being careful not to let them touch and careful to balance evenly around wall.

12. Lock down lid and start timer. Spin for 75 seconds.

13. Unlock lid and remove bags. Weigh each bag and record weight (centrifuge weight).

Calculations:

The tea bag material has an absorbency determined as follows:

Free Swell Capacity, factor=5.78

Centrifuge Capacity, factor=0.50

Z=Oven dry SAP wt (g)/Air dry SAP wt (g)

Free Capacity (g/g):

$$\frac{\begin{matrix}[ ( drip wt ( g ) - dry bag wt ( g ) ) - ( AD SAP wt ( g ) ) ] - \\ ( dry bag wt ( g ) * 5.78 )\end{matrix}}{( AD SAP wt ( g ) * Z )}$$

Centrifuge Capacity (g/g):

$$\frac{\begin{matrix}[ centrifuge wt ( g ) - dry bag wt ( g ) - ( AD SAP wt ( g ) ) ] - \\ ( dry bag wt ( g ) * 0.50 )\end{matrix}}{( AD SAP wt * Z )}$$

### Example 2

### Method for Determining Absorbency Under Load (AUL)

In this example, a method for determining Absorbency Under Load (AUL) is described.

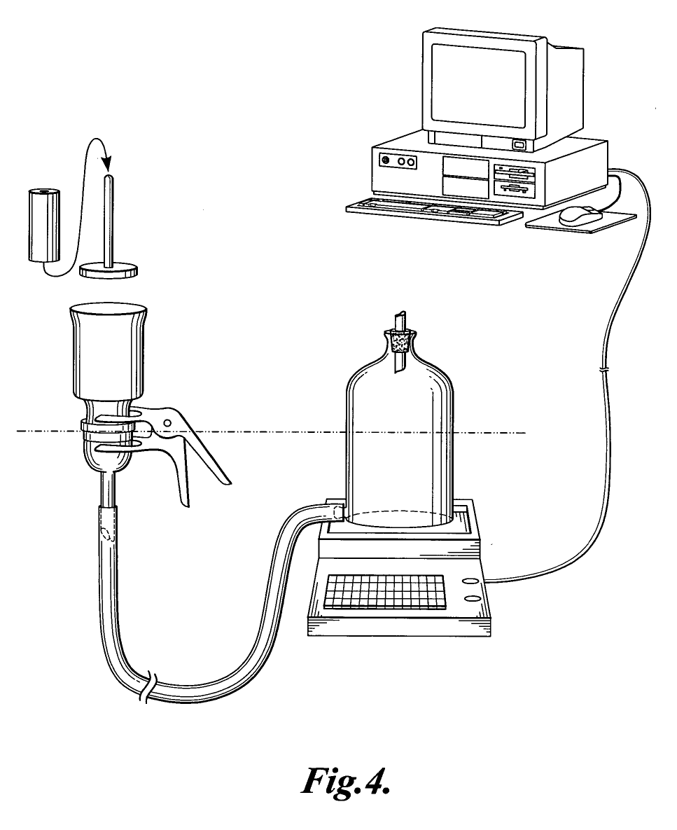

The materials, procedure, and calculations to determine AUL were as follows. Reference is made to FIG. 4.

Test Materials:

Mettler Toledo PB 3002 balance and BALANCE-LINK software or other compatible balance and software. Software set-up: record weight from balance every 30 sec (this will be a negative number. Software can place each value into EXCEL spreadsheet.

Kontes 90 mm ULTRA-WARE filter set up with fritted glass (coarse) filter plate. clamped to stand.

2 L glass bottle with outlet tube near bottom of bottle.

Rubber stopper with glass tube through the stopper that fits the bottle (air inlet).

TYGON tubing.

Stainless steel rod/plexiglass plunger assembly (71 mm diameter).

Stainless steel weight with hole drill through to place over plunger (plunger and weight=867 g)

VWR 9.0 cm filter papers (Qualitative 413 catalog number 28310-048) cut down to 80 mm size.

Double-stick SCOTCH tape.

0.9% Saline.

Test Procedure:

1. Level filter set-up with small level.

2. Adjust filter height or fluid level in bottle so that fritted glass filter and saline level in bottle are at same height.

3. Make sure that there are no kinks in tubing or air bubbles in tubing or under fritted glass filter plate.

4. Place filter paper into filter and place stainless steel weight onto filter paper.

5. Wait for 5-10 min while filter paper becomes fully wetted and reaches equilibrium with applied weight.

6. Zero balance.

7. While waiting for filter paper to reach equilibrium prepare plunger with double stick tape on bottom.

8. Place plunger (with tape) onto separate scale and zero scale.

9. Place plunger into dry test material so that a monolayer of material is stuck to the bottom by the double stick tape.

10. Weigh the plunger and test material on zeroed scale and record weight of dry test material (dry material weight 0.15 g+/−0.05 g).

11. Filter paper should be at equilibrium by now, zero scale.

12. Start balance recording software.

13. Remove weight and place plunger and test material into filter assembly.

14. Place weight onto plunger assembly.

15. Wait for test to complete (30 or 60 min)

16. Stop balance recording software.

Calculations:
A=balance reading (g) * −1 (weight of saline absorbed by test material)
B=dry weight of test material (this can be corrected for moisture by multiplying the AD weight by solids %).
AUL (g/g)=A/B (g 1% saline/1 g test material)

While the preferred embodiment of the invention has been illustrated and described, it will be appreciated that various changes can be made therein without departing from the spirit and scope of the invention.

The embodiments of the invention in which an exclusive property or privilege is claimed are defined as follows:

1. A method for making a composition, comprising reacting a sulfoalkyl cellulose and a synthetic water-soluble polymer with a crosslinking agent, wherein the crosslinking agent reacts with at least one of the sulfoalkyl cellulose or synthetic water-soluble polymer.

2. The method of claim 1, wherein the ratio of sulfoalkyl cellulose to synthetic water-soluble polymer is from about 50:50 to about 95:5 weight/weight.

3. The method of claim 1, wherein the sulfoalkyl cellulose is selected from the group consisting of sulfoethyl cellulose and sulfo-2-hydroxypropyl cellulose.

4. The method of claim 1, wherein the water-soluble polymer is selected from the group consisting of a polyacrylamide, a polyacrylic acid, a polymaleic acid, a polyaspartic acid, a copolymer of acrylic acid and acrylamide, a copolymer of acrylic acid and maleic acid, a copolymer of maleic acid and itaconic acid, and mixtures thereof.

5. The method of claim 1, wherein the crosslinking agent is selected from the group consisting of an aldehyde, a dialdehyde, a dialdehyde sodium bisulfite addition product, a dihalide, a diene, a diepoxide, a haloepoxide, a dicarboxylic acid, a polycarboxylic acid, a diol, a diamine, an aminol, a polyoxazoline functionalized polymer, a polyvalent cation, a polycationic polymer, and mixtures thereof.

6. A method for making a composition, comprising
(a) combining a sulfoalkyl cellulose, a synthetic water-soluble polymer, and a crosslinking agent in an aqueous solution to provide a reaction mixture;
(b) precipitating the reaction mixture by addition of a water-miscible solvent to provide a precipitated mixture;
(c) collecting the precipitated mixture; and
(d) crosslinking the precipitated mixture to provide the composition.

7. The method of claim 6, wherein the ratio of sulfoalkyl cellulose to synthetic water-soluble polymer is from about 50:50 to about 95:5 weight/weight.

8. The method of claim 6, wherein the sulfoalkyl cellulose is selected from the group consisting of sulfoethyl cellulose and sulfo-2-hydroxypropyl cellulose.

9. The method of claim 6, wherein the synthetic water-soluble polymer is selected from the group consisting of a polyacrylamide, a polyacrylic acid, a polymaleic acid, a polyaspartic acid, a copolymer of acrylic acid and acrylamide, a copolymer of acrylic acid and maleic acid, a copolymer of maleic acid and itaconic acid, and mixtures thereof.

10. The method of claim 6, wherein the crosslinking agent is selected from the group consisting of an aldehyde, a dialdehyde, a dialdehyde sodium bisulfite addition product, a dihalide, a diene, a diepoxide, a haloepoxide, a dicarboxylic acid, a polycarboxylic acid, a diol, a diamine, an aminol, a polyoxazoline functionalized polymer, a polyvalent cation, a polycationic polymer, and mixtures thereof.

11. The method of claim 6 further comprising combining the sulfoalkyl cellulose, the synthetic water-soluble polymer, and the crosslinking agent with a second crosslinking agent.

12. The method of claim 6, wherein the second crosslinking agent is different from the crosslinking agent combined with the sulfoalkyl cellulose and the water-soluble polymer in step (a).

13. The method of claim 11, wherein the second crosslinking agent is selected from the group consisting of an aldehyde, a dialdehyde, a dialdehyde sodium bisulfite addition product, a dihalide, a diene, a diepoxide, a haloepoxide, a dicarboxylic acid, a polycarboxylic acid, a diol, a diamine, an aminol, a polyoxazoline functionalized polymer, a polyvalent cation, a polycationic polymer, and mixtures thereof.

14. A method for making a composition, comprising:
(a) treating a sulfoalkyl cellulose and a synthetic water-soluble polymer with a crosslinking agent to provide a reaction mixture; and
(b) crosslinking the reaction mixture to provide the composition.

15. The method of claim 14, wherein the ratio of sulfoalkyl cellulose to synthetic water-soluble polymer is from about 50:50 to about 95:5 weight/weight.

16. The method of claim 14, wherein the sulfoalkyl cellulose is selected from the group consisting of sulfoethyl cellulose and sulfo-2-hydroxypropyl cellulose.

17. The method of claim 14, wherein the synthetic water-soluble polymer is selected from the group consisting of a polyacrylamide, a polyacrylic acid, a polymaleic acid, a polyaspartic acid, a copolymer of acrylic acid and acrylamide, a copolymer of acrylic acid and maleic acid, a copolymer of maleic acid and itaconic acid, and mixtures thereof.

18. The method of claim 14, wherein the crosslinking agent is selected from the group consisting of an aldehyde, a dialdehyde, a dialdehyde sodium bisulfite addition product, a dihalide, a diene, a diepoxide, a haloepoxide, a dicarboxylic acid, a polycarboxylic acid, a diol, a diamine, an aminol, a polyoxazoline functionalized polymer, a polyvalent cation, a polycationic polymer, and mixtures thereof.

19. The method of claim 14 further comprising combining the sulfoalkyl cellulose, the synthetic water-soluble polymer, and the crosslinking agent with a second crosslinking agent.

20. The method of claim 19, wherein the second crosslinking agent is different from the crosslinking agent combined with the sulfoalkyl cellulose and the synthetic water-soluble polymer in step (a).
